(19) United States
(12) Patent Application Publication
(10) Pub. No.: US 2020/0046232 A1
(43) Pub. Date: Feb. 13, 2020

(54) BLOOD CIRCULATION DETECTION APPARATUS AND BLOOD CIRCULATION DETECTION METHOD

(71) Applicants: Kabushiki Kaisha Toshiba, Tokyo (JP); Toshiba Electronic Devices & Storage Corporation, Tokyo (JP)

(72) Inventor: Ken Kawakami, Kawasaki Kanagawa (JP)

(21) Appl. No.: 16/299,503

(22) Filed: Mar. 12, 2019

(30) Foreign Application Priority Data
Aug. 10, 2018 (JP) 2018-151989

(51) Int. Cl. A61B 5/0285; A61B 5/00; A61B 5/021; A61B 5/024

(52) U.S. Cl. CPC A61B 5/0285; A61B 5/7239; A61B 5/02116; A61B 5/0402; A61B 5/681; A61B 5/7275; A61B 5/02427

(57) ABSTRACT

A blood circulation detection apparatus has a measurer to measure a pulse wave of a subject based on a received optical signal diffused in a body of the subject and received when an optical signal in a predetermined frequency band is emitted to the subject, and a blood circulation detector to detect blood circulation of the subject based on a D. C. component of the received optical signal, a blood-volume change amount of the pulse wave from a rising time of the pulse wave to a time when a first-order differentiation value of the pulse wave with time becomes maximum, and a first-order differentiation value of the blood volume change amount with time.

### CROSS REFERENCE TO RELATED APPLICATIONS

This application is based upon and claims the benefit of priority from the prior Japanese Patent Application No. 2018-151989, filed on Aug. 10, 2018, the entire contents of which are incorporated herein by reference.

### FIELD

Embodiments of the present disclosure relate to a blood circulation detection apparatus and a blood circulation detection method.

### BACKGROUND

A photoplethysmogram (PPG) sensor, which measures the change in blood volume in arteries and capillary vessels corresponding to the change in heart rate to detect pulse waves in accordance with heartbeat, has been known. A method of using the PPG sensor to detect the heart rate based on the blood volume passing through tissue per heart rate is referred to as a blood volume pulse (BVP) measurement.

The PPG sensor can be used for the purpose of detecting various biometric information other than the heart rate. Blood circulation, which is one of the biometric information, is the state of blood flow and generally evaluated with a blood flow velocity and a blood flow amount by a blood flow sensor. It is considered that, the smaller the degree of change in blood flow in accordance with heart beating, the more evenly blood goes around, so that it is determined to be a good state of blood circulation.

However, since the blood flow sensor is not built in a general fitness tracker and the like, it is an actual situation that there is no means for easily monitoring the change in blood circulation on a daily basis.

### BRIEF DESCRIPTION OF THE DRAWINGS

FIG. 1 a block diagram schematically showing the configuration of a blood circulation detection apparatus according to an embodiment;

FIG. 2 is a figure showing an example of a wristwatch-type biometric measuring apparatus;

FIG. 3 is an example of the waveform of a pulse wave;

FIG. 4A is a figure showing a waveform of an optical signal received by a photoreceptor;

FIG. 4B is a figure showing a waveform of a pulse wave generated by a pulse wave generator;

FIG. 5 is a graph showing the relationship between blood circulation, and a mean blood pressure and a mean blood flow velocity; and

FIG. 6 a block diagram schematically showing the configuration of a pulse wave evaluation apparatus for evaluating a pulse wave.

### DETAILED DESCRIPTION

According to one embodiment, a blood circulation detection apparatus has a measurer to measure a pulse wave of a subject based on a received optical signal diffused in a body of the subject and received when an optical signal in a predetermined frequency band is emitted to the subject, and a blood circulation detector to detect blood circulation of the subject based on a D. C. component of the received optical signal, a blood-volume change amount of the pulse wave from a rising time of the pulse wave to a time when a first-order differentiation value of the pulse wave with time becomes maximum, and a first-order differentiation value of the blood volume change amount with time.

Hereinafter, an embodiment will now be explained with reference to the accompanying drawings. In the following embodiment, a unique configuration and operation of a blood circulation detection apparatus will be mainly explained. However, the pulse wave evaluation apparatus may have other configurations and operations omitted in the following explanation.

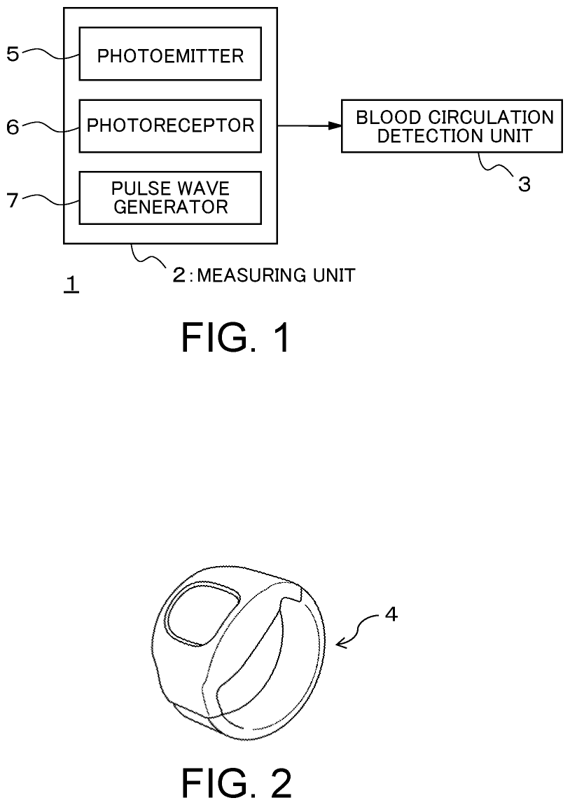

FIG. 1 a block diagram schematically showing the configuration of a blood circulation detection apparatus 1 according to an embodiment. The blood circulation detection apparatus 1 is provided with a measuring unit (measurer) 2 and a blood circulation detection unit (blood circulation detector) 3. The blood circulation apparatus 1 may, for example, be built in a wristwatch-type biometric measuring apparatus 4 such as shown in FIG. 2.

The measuring unit 2 measures the change in blood volume of arteries and capillary vessels in accordance with the change in heart rate of a subject to acquire information on the blood volume pulse in accordance with heartbeat. Hereinafter, the blood volume pulse is simply referred to as a pulse wave as required.

The measuring unit 2 has a photoemitter 5, a photoreceptor 6, and a pulse wave generator 7. The photoemitter 5 has, for example, an LED (Light Emitting Diode) that emits an optical signal in a predetermined wavelength band (green, near-infrared band, etc.). The photoreceptor 6 receives a signal that is the optical signal from the photoemitter 5, after reflected and diffused in the body of a subject. The pulse wave generator 7 generates a PPG signal per one beat of heartbeat based on the optical signal received by the photoreceptor 6. The PPG signal includes information on light intensity of a diffused light component that has not been absorbed by the tissue of the subject including blood vessels.

When the emission amount of the optical signal from the photoemitter 5 varies, the reception amount of the signal at the photoreceptor 6 also varies. For this reason, the pulse wave generator 7 separates the received optical signal into a D. C. component and an A. C. component, and generates a pulse wave based on the A. C./D. C. ratio. Therefore, the generated pulse wave is non-dimensional data.

The blood circulation detection unit 3 detects blood circulation of the subject based on the D. C. component of the received optical signal, a blood-volume change amount of the pulse wave from a rising time of the pulse wave to a time at which a value, which is obtained by differentiating the pulse wave with time by first-order differentiation, becomes maximum, and a value obtained by differentiating the blood-volume change amount with time by first-order differentiation.

A maximum flow velocity vₘₐₓ in the case where a blood flow in a blood vessel is assumed by Hagen-Poiseuille flow is expressed by the following expression (1).

$${v}_{max} = - \frac{dp}{dz} \cdot \frac{{r}^{2}}{4 e} \qquad ( 1 )$$

Here, z is the direction of a blood flow, dp/dz is a pressure gradient to the direction of a horizontal flow, r is the radius of a cylindrical pipe corresponding to a blood vessel, and e is a viscosity constant.

The radius r of a blood vessel varies in inverse proportion to volumetric strain. Therefore, r²/4e in the expression (1) and the volumetric strain can be expressed by the following expression (2).

$$\frac{{r}^{2}}{4 e} = a \frac{x}{{x}^{′}} \qquad ( 2 )$$

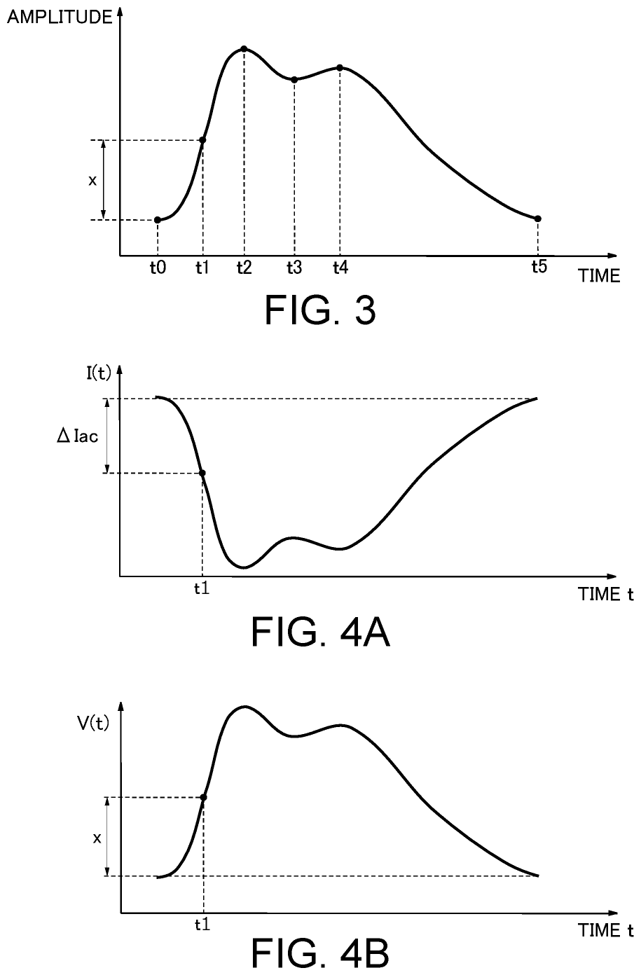

FIG. 3 is an example of the waveform of a normal pulse wave per one beat. In the expression (2), “a” is a constant, x is a value of blood volume pulse from a pulse-wave rising time (t0) to a time (t1) at which a maximum differential coefficient is given, shown in FIG. 3, and x′ is a time derivative of x (a value obtained by differentiating x with time by first-order differentiation). From the expressions (1) and (2), the following expression (3) holds when a flow velocity at x=1 is defined as a criterial flow velocity v_{criterion}.

$$v - {v}_{criterion} = a \frac{dp}{dz} \cdot ( \frac{1}{{x}^{′}} - \frac{x}{{x}^{′}} ) \qquad ( 3 )$$

In the expression (3), the term inside the parenthesis in the right side is an indicator indicating blood circulation PCI shown in the following expression (4).

$$PCI = \frac{1}{{x}^{′}} - \frac{x}{{x}^{′}} \qquad ( 4 )$$

In general, since a measured value of blood volume pulse is affected by the optical signal from the photoemitter 5, it is required to use a ratio of A. C. and D. C. components. According to a blood-volume change expressing method (mNPV: the modified Normalized Pulse Volume) by means of blood volume pulse using the Beer Lambert law, the blood volume change (blood volume pulse) x is expressed by the following expression (5).

$$x ∝ \frac{Δ {I}_{a c}}{{I}_{d c}} \qquad ( 5 )$$

The sign I_{dc} is a D. C. component of the received optical signal and ΔI_{ac} is an D. C. component of the received optical signal. Using the expression (5), the expression (4) can be expressed as the following expression (6).

$${PCI}_{1} = \frac{{I}_{d c} - Δ {I}_{a c}}{{( Δ {I}_{a c} )}^{′}} \qquad ( 6 )$$

FIG. 4A is a figure showing a waveform of the received optical signal at the photoreceptor 6. FIG. 4B is a figure showing a waveform of the pulse wave generated by the pulse wave generator 7. In FIGS. 4A and 4B, the abscissa is time and the ordinate is current and voltage, respectively. In the expression (6), ΔI_{ac} is the blood-volume change amount from a received optical-signal rising time (t0) to a time (t1) at which a maximum differential coefficient is given, and (ΔI_{ac})′ is a derivative of ΔI_{ac} to time.

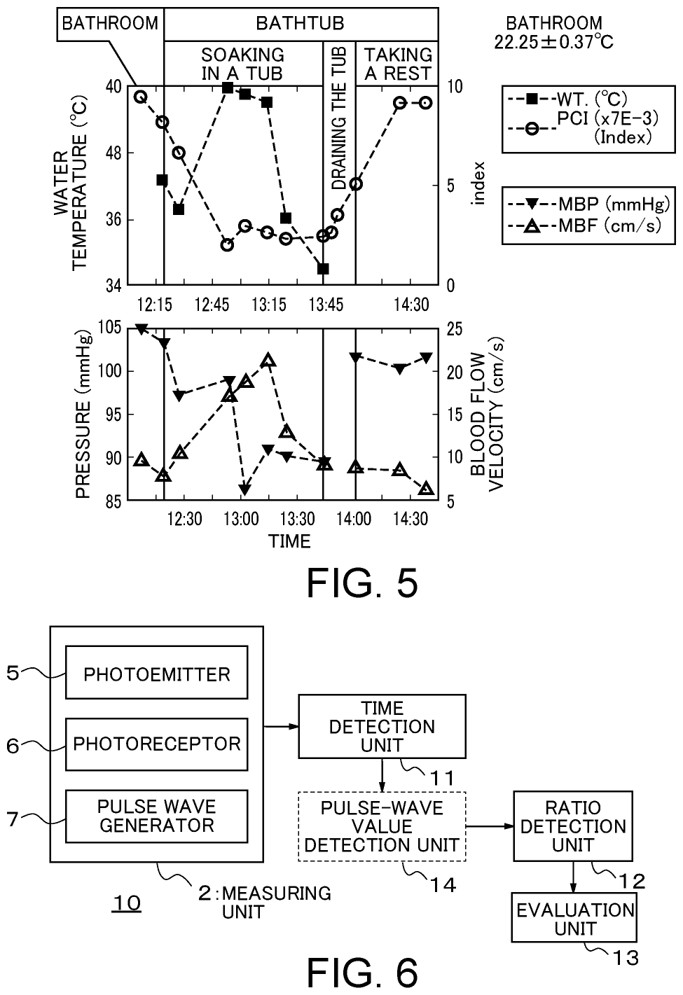

In the present embodiment, the blood circulation PCI is obtained based on the expression (6). FIG. 5 is a graph showing the relationship between the blood circulation PCT1 calculated by the expression (6), and a mean blood pressure MBP (mmHg) and a mean blood flow velocity MBF (cm/s). This graph shows the change in blood circulation, mean blood pressure and mean blood flow velocity in the case where a subject soaks in a tub for 90 minutes and then takes a rest after draining the tub. Moreover, the graph shows an example of experiment in which, while the subject is soaking in the tub, the temperature of hot water is raised from 36° C. to 40° and then decreased to 34° C.

According to FIG. 5, it is found that although the blood circulation PCT1 has positive correlation with the mean blood pressure, the blood circulation PCT1 does not depend only on the mean blood pressure.

The blood circulation detection apparatus 1 detects blood circulation using a measured pulse wave. However, it is known that the waveform of pulse wave largely varies depending on the active or metal state of a subject. Therefore, the blood circulation may be detected by evaluating in advance whether pulse waves are irregular and using a regular pulse wave.

FIG. 6 a block diagram schematically showing the configuration of a pulse wave evaluation apparatus 10 for evaluating a pulse wave. The pulse wave evaluation apparatus 10 is provided with a measuring unit (measurer) 2, a time detection unit (time detector) 11, a ratio detection unit (ratio detector) 12, and an evaluation unit 13. The pulse wave evaluation apparatus 10 may also be built in, for example, a wristwatch-type biometric measuring apparatus 4 such as shown in FIG. 3. The measuring unit 2 of FIG. 6 may be identical to the measuring unit 2 of FIG. 1.

The time detection unit 11 detects, per one beat of a pulse wave, a rising time of the pulse wave, a time at which a value, which is obtained by differentiating the pulse wave with time by first-order differentiation, becomes maximum, and a time at which the amplitude of the pulse wave becomes a maximum peak. The normal pulse wave shown in FIG. 3 shows change in such a manner to begin at a position (t0) of the bottom of amplitude, reach a maximum amplitude peak (t2) with almost monotonic increase, thereafter, reach the second bottom of amplitude (t3) with monotonic decrease, reach the second amplitude peak (t4) again with monotonic increase, and reach the bottom value (t5) with monotonic decrease to complete.

The time detection unit 11 detects t0 of FIG. 3 as the rising time and detects t2 as the time at which the amplitude becomes the maximum peak. Moreover, the time detection unit 3 detects t1 that is the time at which the value, which is obtained by differentiating the pulse wave with time by first-order differentiation, becomes maximum, between t0 and t2.

The ratio detection unit 12 detects an acceleration ratio of mean acceleration of the pulse wave from the rising time t0 to the time t1 at which the value, which is obtained by differentiating the pulse wave with time by first-order differentiation, becomes maximum, and mean acceleration of the pulse wave from the time t1 to the maximum amplitude time t2.

The evaluation unit 13 evaluates the pulse wave based on the detected acceleration ratio. Since it is not easy to directly detect the mean acceleration between t0 to t1 and t1 to t2 of the pulse wave, the evaluation unit 13 evaluates the pulse wave based on a ratio of an amplitude value x(t2) of the pulse wave at t2 and an amplitude value x(t1) of the pulse wave at t1. In more specifically, the evaluation unit 13 determines whether the pulse wave is irregular per one beat depending on the degree of variation in the ratio.

The blood circulation detection apparatus 1 may be provided with a pulse-wave value detection unit (pulse-wave value detector) 14. The pulse-wave value detection unit 14 detects a value of the pulse wave at the maximum amplitude time t2 and a value of the pulse wave at the time t1 at which the value obtained by first-order differentiation becomes maximum. The ratio detection unit 12 can detect the acceleration ratio based on a pulse-wave value ratio of the two values detected by the pulse-wave value detection unit 14.

The blood circulation detection apparatus 1 may detect blood circulation using a pulse wave that is determined as regular by the pulse wave evaluation apparatus 10. In more specifically, the blood circulation detection apparatus 1 may detect the blood circulation based on the pulse wave determined that the acceleration ratio is equal to or less than a predetermined value.

As described above, in the present embodiment, since blood circulation of a subject is detected based on the expression (6), the blood circulation can be detected easily and accurately. Moreover, by detecting the blood circulation using a regular pulse wave, the blood-circulation detection accuracy can be improved.

At least part of the blood circulation detection apparatus 1 described above may be configured with hardware or software. When it is configured with software, a program that performs at least part of the blood circulation detection apparatus 1 may be stored in a storage medium such as a flexible disk and CD-ROM, and then installed in a computer to run thereon. The storage medium may not be limited to a detachable one such as a magnetic disk and an optical disk but may be a standalone type such as a hard disk and a memory.

Moreover, a program that achieves the function of at least part of the blood circulation detection apparatus 1 may be distributed via a communication network a (including wireless communication) such as the Internet. The program may also be distributed via an online network such as the Internet or a wireless network, or stored in a storage medium and distributed under the condition that the program is encrypted, modulated or compressed.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A blood circulation detection apparatus comprising:
a measurer to measure a pulse wave of a subject based on a received optical signal diffused in a body of the subject and received when an optical signal in a predetermined frequency band is emitted to the subject; and
a blood circulation detector to detect blood circulation of the subject based on a D. C. component of the received optical signal, a blood-volume change amount of the pulse wave from a rising time of the pulse wave to a time when a first-order differentiation value of the pulse wave with time becomes maximum, and a first-order differentiation value of the blood volume change amount with time.

2. The blood circulation detection apparatus of claim 1, wherein the blood circulation detector detects the blood circulation based on a value obtained by dividing a value, obtained by subtracting the blood-volume change amount of the pulse wave from the D. C. component of the received optical signal, by the first-order differentiation value of the blood-volume change amount with time.

3. The blood circulation detection apparatus of claim 1 further comprising:
a time detector to detect, per one beat of the measured pulse wave, a rising time of the pulse wave, a time of the first-order differentiation value of the pulse wave with time, and a maximum amplitude time of the pulse wave;
a ratio detector to detect an acceleration ratio of mean acceleration from the rising time to the time when the first-order differentiation value of the pulse wave with time becomes maximum and mean acceleration from the time when the first-order differentiation value of the pulse wave with time becomes maximum to the maximum amplitude time; and
an evaluator to evaluate the pulse wave based on the acceleration ratio.

4. The blood circulation detection apparatus of claim 3, wherein the blood circulation detector detects the blood circulation based on the pulse wave that the acceleration ratio is equal to or less than the predetermined threshold value.

5. A blood circulation detection method to be executed on computer comprising:
measuring a pulse wave of a subject based on a received optical signal diffused in a body of the subject and received when an optical signal in a predetermined frequency band is emitted to the subject; and
detecting blood circulation of the subject based on a D. C. component of the received optical signal, a blood-volume change amount of the pulse wave from a rising time of the pulse wave to a time when a first-order differentiation value of the pulse wave with time becomes maximum, and a first-order differentiation value of the blood-volume change amount with time, or based on a value obtained by dividing the blood-volume change amount of the pulse wave by a first-order differentiation value of the blood-volume change amount with time.

6. The blood circulation detection method of claim 5, wherein the blood circulation is detected based on a value obtained by dividing a value obtained by subtracting the blood-volume change amount of the pulse wave from the D. C. component of the received optical signal, by the first-order differentiation value of the blood-volume change amount with time.

7. The blood circulation detection method of claim 5, wherein the blood circulation is detected based on a pulse wave determined that an acceleration ratio of mean acceleration from the rising time to the time when the first-order differentiation value of the pulse wave with time becomes maximum and mean acceleration from the time when the first-order differentiation value of the pulse wave with time becomes maximum to the maximum amplitude time is equal to or less than a predetermined threshold value.

8. The blood circulation detection method of claim 5 further comprising:
detecting, per one beat of the measured pulse wave, a rising time of the pulse wave, a time of the first-order differentiation value of the pulse wave with time becomes maximum, and a maximum amplitude time of the pulse wave;
detecting an acceleration ratio of mean acceleration from the rising time to the time when the first-order differentiation value of the pulse wave with time becomes maximum and mean acceleration from the time when the first-order differentiation value of the pulse wave with time becomes maximum to the maximum amplitude time; and
evaluating the pulse wave based on the acceleration ratio.
